(19) United States
(12) Patent Application Publication
(10) Pub. No.: US 2025/0059487 A1
(43) Pub. Date: Feb. 20, 2025

(54) METHOD OF INJECTION OF CO2 AND ELIMINATION OF O2 IN A PLATE-TYPE REACTOR

(71) Applicant: FERMENTALG, Libourne (FR)

(72) Inventor: Philippe BOUDIER, Cenon (FR)

(21) Appl. No.: 18/721,948

(22) PCT Filed: Dec. 21, 2022
(86) PCT No.: PCT/EP2022/087329

§ 371 Date: Jun. 20, 2024

(30) Foreign Application Priority Data
Dec. 21, 2021 (FR) FR2114060

(51) Int. Cl. C12M 1/00; C12M 1/34

(52) U.S. Cl. CPC C12M 29/16; C12M 21/02; C12M 29/04; C12M 41/34

(57) ABSTRACT

The present invention concerns a method of cultivation of microorganisms in a bioreactor wherein the culture medium comprises carbon dioxide (CO₂) as carbon source. The CO₂ supply in the bioreactor is regulated using a membrane contactor.

### FIELD OF THE INVENTION

The present invention concerns a method of cultivation of microorganisms in a bioreactor wherein the culture medium comprises carbon dioxide (CO₂) as carbon source. The CO₂ supply in the bioreactor is regulated using a membrane contactor.

### STATE OF THE ART

The methods of cultivation of microorganisms are well known to the person skilled in the art. The cultivation may be undertaken in the laboratory in Petri dishes or at industrial scale in bioreactors. The cultivation of microorganisms requires reproducing the conditions of the original environment for optimal growth. Thus, the presence of organic substrates is essential. Other parameters must also be reproduced, such as light, temperature, pressure, humidity, pH, and gas concentrations.

Many microorganisms use carbon dioxide as carbon source. In particular, autotrophic cultures do not include any source of carbon other than the CO₂ contained in the air. The CO₂ is transformed into dioxygen (O₂) and eliminated in the atmosphere. However, it has been found that an excessive concentration of CO₂ leads to acidification of the culture medium and a reduction in the pH. In parallel, an excessive quantity of O₂ in the culture medium leads to decreased growth of the microorganisms, and fosters the development of bacteria. Regulation of the CO₂ and O₂ concentration in the culture media of the microorganisms is therefore essential to their healthy development.

There are many devices that regulate the flows of CO₂ and O₂, such as membranes (Y. S. Lai et al., Algal Research 2020, 52, 102098; US 2021/053012), membrane bubblers and bubble columns, ceramic bubblers, and even bubble diffuser stones, etc. In spite of the fact that these methods present a good rate of transfer, losses of matter are observed. Moreover, these devices often lead to phenomena of coalescence on the walls of the bioreactor, the blades, the light devices or the bubblers, leading notably to the obstruction of the air/gas inlets and a reduction in the light supply.

Furthermore, when the desorption of the O₂ is undertaken via periodic agitation (sequences of large bubbles, gentle mechanical agitation with banana blades), it is necessary to send a strong airflow with little CO₂ (98% air) in order to obtain appropriate dissolved CO₂ concentrations, which accentuates the shear constraints in the medium.

It would be desirable to be able to avoid these phenomena which disrupt the development of the strains. There is therefore a need to develop a method of cultivation of microorganisms where the flows of CO₂ and O₂ are optimally regulated, and where the problems linked to the use of the existing devices are minimal, even non-existent.

Given the environmental issues linked to the production of CO₂ released into the atmosphere, notably linked to the production of CO₂-rich gas, such as gaseous industrial effluents, it has been envisaged to use these gases to supply bioreactors for the growth of microorganisms capable of growing with the CO₂ as sole carbon source. This depollution of the gas, also called decarbonation, is notably described in patent applications WO2018/060197, WO2019/101899 and WO2021/165621. While these methods have shown a good level of efficacy in consuming the CO₂ provided by the gases to be decarbonated, they still need to be improved so as to avoid some of the unconsumed CO₂ being released into the atmosphere.

The inventors have thus developed a method of cultivation of microorganisms comprising CO₂ as carbon source and comprising a step of cultivation of said microorganism in a bioreactor making it possible to improve the CO₂ supply, particularly for the decarbonation of industrial gases.

### PRESENTATION OF THE INVENTION

The present invention concerns a method of cultivation of microorganisms comprising CO₂ as carbon source and comprising a step of cultivation of said microorganism in a bioreactor comprising CO₂ supply means, and characterised in that the CO₂ supply means comprise a membrane contactor to regulate the flows of CO₂ and O₂ in the culture medium, the flow of CO₂ being directed to the culture medium, and the flow of O₂ being directed to the exterior of the culture medium.

Within the framework of this invention, the microorganisms are mainly bacteria, yeasts and protists.

The method of cultivation advantageously comprises the following steps:
(a) Cultivation of the microorganisms; and
(b) Recovery of the biomass produced by said microorganisms.

The invention also concerns the use of a membrane contactor to regulate the flows of CO₂ and O₂ in a method of cultivation of microorganisms wherein the CO₂ is a carbon source in the culture medium.

The method of cultivation according to the invention is particularly suitable for the decarbonation of a CO₂-rich gas.

The invention also concerns a method of decarbonation of a CO₂-rich gas which comprises the injection of said gas in a bioreactor, said bioreactor comprising a culture medium and microorganisms capable of growing in said culture medium with the CO₂ as sole carbon source and CO₂ supply means, characterised in that the CO₂ supply means comprise a membrane contactor to regulate the flows of CO₂ and O₂ in the culture medium, the flow of CO₂ being directed to the culture medium and the flow of O₂ being directed to the exterior of the culture medium.

Lastly, the invention concerns a microalgae fermentation device comprising a fermentation reactor, CO₂ supply means, and where applicable lighting means, characterised in that the CO₂ supply means comprise a membrane contactor to regulate the flows of CO₂ and O₂, wherein the flow of CO₂ is directed to the culture medium and the flow of O₂ is directed to the exterior of the culture medium.

### BRIEF DESCRIPTION OF THE FIGURES

FIG. 1 represents a method of cultivation of microorganisms in the presence of a membrane contactor.

FIG. 2 represents a membrane contactor according to the invention (FIG. 2A) and a cross-section of a transfer membrane of the membrane contactor (FIG. 2B).

FIGS. 3A and 3B are representations of the phenomena occurring in the transfer membrane of the membrane contactor according to the invention, notably phenomena of diffusion (FIG. 3A) and flows of carbon dioxide (CO₂), dioxygen (O₂) and the culture medium (FIG. 3B).

FIG. 4 represents the speed of the medium in the membrane contactor according to the productivity of the biomass determined at 0.2 kg/m³/J.

FIG. 5 represents the speed of the medium in the membrane contactor according to the productivity of the biomass determined at 0.79 kg/m³/J.

FIG. 6 represents the variation in CO₂ pressure in the transfer membrane of the membrane contactor according to the quantity of CO₂ in the culture medium.

FIG. 7 represents the quantity of CO₂ to be injected into the culture medium according to the temperature of the medium and the pressure in the transfer membrane of the membrane contactor.

FIG. 8 is a diagrammatic representation of a membrane contactor according to one embodiment of the invention.

FIG. 9 is a graph showing the evolution over time of the O₂ pressure (PO2) in the culture medium.

FIG. 10 is a graph presenting the optical density (OD) results and daily productivity (based on the OD) of Chlorella sorokiniana cultivated in a bioreactor equipped with a membrane contactor in compliance with the invention, compared with the results obtained with Chlorella sorokiniana cultivated in a control bioreactor not comprising a membrane contactor.

### DETAILED DESCRIPTION OF THE INVENTION

The present invention concerns a method of cultivation of microorganisms in a culture medium comprising carbon dioxide (CO₂) as carbon source and comprising a step of cultivation of the microorganism in a bioreactor comprising CO₂ supply means, characterised in that the CO₂ supply means comprise a membrane contactor to regulate the flows of CO₂ and dioxygen (O₂) in the culture medium, and wherein the flow of CO₂ is directed to the culture medium and the flow of O₂ is directed to the exterior of the culture medium.

Methods of cultivation of microorganisms are well known to the person skilled in the art, whether in heterotrophic, autotrophic or mixotrophic mode.

The cultivation of microorganisms involves many parameters such as the nature and the composition of the culture medium itself, but also external factors which must be controlled and regulated throughout the process, such as light, temperature, pressure, humidity, pH, gas concentrations in the reactor, etc.

Culture media, essential to the development of the microorganisms, are well known to the person skilled in the art, who will be able to determine and adapt the quantity and the nature of substrates for their cultivation, such as for example the sources of carbon, nitrogen, oxygen, phosphorus, etc.; the minerals, such as calcium, iron, magnesium, potassium, sodium, sulphur, etc.; and trace elements (boron, zinc, copper, cobalt, etc.); according to the microorganism(s) cultivated, the size of the reactor, the quantity of biomass desired, etc.

The invention is particularly suitable for methods of cultivation in a bioreactor. Within the framework of this invention, bioreactor means a reactor within which biological phenomena develop, such as growth of pure cultures or microorganisms or of a consortium of microorganisms, in highly varied sectors such as the treatment of effluents or the production of biomass containing molecules of interest.

Bioreactors are well known to the person skilled in the art, and generally comprise a closed tank in which a stirring or agitation element is fitted, intended to foster the homogenisation of the content of the tank. The content of the tank may also be stirred or agitated via a liquid pump which induces a flow of circulation of the culture medium within the tank.

The invention is particularly suitable for cultivation in autotrophic mode, said cultivation comprising carbon dioxide (CO₂) as carbon source and light source. The method of cultivation then also comprises an illumination stage.

Bioreactors may also comprise illumination means, where the light supply can be provided continuously or cyclically (WO2019/034792, WO2021/160776). It is understood within the framework of this invention that the person skilled in the art knows to adapt the speed of agitation and the quantity of light in order to obtain optimal cultivation conditions.

Bioreactors also generally comprise an air inlet and outlet enabling a gas exchange between the outside and the inside of the reactor.

The air inlet may thus be coupled with a device, such as a pump, in order to inject a gas into the culture medium. The gas injected is chosen from among the gases necessary to the development of the microorganisms in cultivation in said bioreactor. They are notably carbon dioxide (CO₂), carbon monoxide (CO), dioxygen (O₂) or dinitrogen (n2), or a mixture thereof. In a preferred embodiment, the gas injected into the culture medium is carbon dioxide.

The carbon dioxide injected into the reactor is supplied via an ambient airflow or via an external source such as industrial gases, fermentation processes or gas cylinders.

The CO₂ is thus dissolved in the culture medium and consumed by the microorganisms, while O₂ is produced. This O₂ is eliminated via the air outlet of the bioreactor.

It should be noted that the reactors may be equipped with other devices or apparatus used to control and regulate the reactor and the growth of the microorganisms. These may particularly be sensors or probes to measure the different cultivation parameters, such as for example: temperature, pH, pressure on entering and leaving the reactor, etc.

According to the invention, in order to regulate the gas flow rates, and notably the flow of CO₂ inside the bioreactor, one or more membrane contactors is used.

A membrane contactor is a device comprising at least two fluid circulation circuits separated by a transfer membrane. The fluids circulating in said membrane contactor are also called circulation fluids. The membrane contactor thus comprises a first circulation circuit of a first circulation fluid supplying the carbon dioxide (CO₂), called CO₂ supply fluid, in contact with a first side of the transfer membrane, and a second circuit for circulation of a second circulation fluid for receiving the CO₂, called CO₂ receiving fluid, in contact with the second side of the transfer membrane. Each fluid circulation circuit is independent of each other, and can be closed or open.

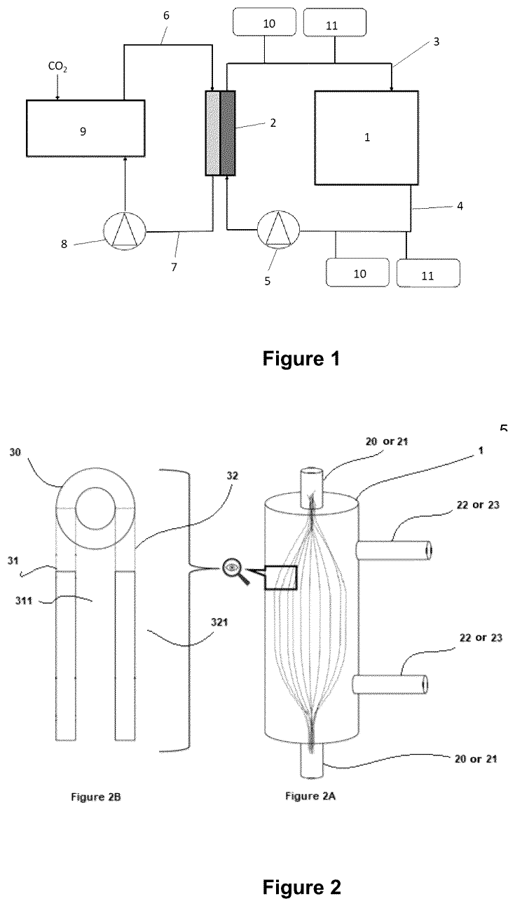

In one embodiment, the circulation circuit of the CO₂ receiving fluid is a closed circuit. In another embodiment, the circulation circuit of the CO₂ receiving fluid and the circulation circuit of the CO₂ supply fluid are closed circuits in order to ensure optimal conditions of growth of the microorganisms. In reference to FIG. 1, the circuit of the CO₂ receiving fluid (3) and (4) and the circuit of the CO₂ supply fluid (6) and (7) are closed circuits where the exchanges between the two fluids take place in the membrane contactor (2).

The CO₂ supply fluid is in the form of gas, preferentially an air/CO₂ mixture comprising at least 5% CO₂, even at least 25% CO₂, further preferentially at least 50% CO₂, and even further preferentially at least 75% CO₂. Advantageously, the supply fluid is of gaseous CO₂ with a content of 100% CO₂.

The CO₂ receiving fluid is preferentially in the liquid form according to the invention. This is more specifically the culture medium. In this case, the CO₂ receiving fluid comprises the microorganisms and the nutrients necessary to their growth.

Thus, according to the invention, the membrane contactor makes it possible to ensure the exchange of CO₂ molecules between a CO₂ supply fluid, preferably in the form of gas, and the CO₂ receiving fluid, which is the culture medium, through a membrane, called the transfer membrane.

Within the membrane contactor, the two fluids, the CO₂ supply fluid and the CO₂ receiving fluid, are in circulation.

The circulation of the CO₂ supply fluid can be assured by all means known to the person skilled in the art, notably by using a CO₂ source that is already pressurised, or using a gas compressor which makes it possible to ensure a regular flow in the membrane contactor. In parallel, the circulation of the CO₂ receiving fluid, i.e. the culture medium, is allowed thanks to the rotation of the blades in the bioreactor or via the pumping of the culture medium which is then reinjected into the bioreactor using a pump. A vacuum pump id preferentially used.

Advantageously, the use of a membrane contactor according to the invention enables a method of injection of the CO₂ which does not generate harmful shear constraints in the reactor, which avoids the formation of foam in the culture.

In one embodiment, the flows of the CO₂ supply fluid and the CO₂ receiving fluid are parallel. In another embodiment, the flows are perpendicular. In yet another embodiment, the flows are crossed. Preferentially, the flows of the CO₂ supply fluid and the CO₂ receiving fluid are crossed, i.e. the CO₂ supply fluid and the CO₂ receiving fluid circulate in opposite directions.

In reference to FIG. 2, the membrane contactor (1) then comprises an inlet (20) or (21) and an outlet (20) or (21) for a first circulation fluid, and an inlet (22) or (23) and an outlet (22) or (23) for a second circulation fluid. These two fluids are separated by a transfer membrane (30). The membrane contactor then also comprises two circulation circuits: a first circulation circuit of a first circulation fluid in contact with a first side of the membrane, and a second circulation circuit of the second circulation fluid in contact with a second side of the membrane.

The membrane therefore makes it possible to promote the contact between the two circulation fluids without having direct physical contact. The properties of the membrane determine the nature and the characteristics of the exchanges between the two circulation fluids.

The membrane may be a porous or non-porous membrane, also called dense or pervaporation membrane. Dense membranes are membranes without created porosity. They are divided into two categories: hydrophilic membranes and organophilic membranes.

Hydrophilic membranes enable water to pass through, and therefore, for example, the dehydration of a solvent. Typically, these membranes are made of a polymer material (polyvinyl alcohol) deposited on a porous medium. Their geometric features are identical to those of ultrafiltration membranes, for example.

These membranes are implemented mainly in flat modules, the “permeate” compartment being put into contact with a partial vacuum chamber, so as to maintain the agitating force and evacuate the products of the separation.

Organophilic membranes are mainly silicone-based (PDMS polydimethylsiloxane) and, in this case, it is the affinity of the silicone material for one or more of the compounds of the mixture which enable a selective transfer across the membrane. Very high selectivity is thus obtained, with the transfer flows generally remaining weak.

Membrane contactors are characterised by the transfer membranes composing them, more specifically by the geometric configuration and the structure of those membranes.

Membranes may be of flat, spiral, tubular or hollow fibre geometric configuration (i.e. pipes or tubes).

According to a preferred embodiment, the membrane(s) have a hollow fibre geometric configuration. Advantageously, the membrane contactor comprises several fibres dispersed in bundles of tubes. The external diameter of the membranes is greater than 50 μm, preferentially greater than 60 μm, even more preferentially greater than 70 μm. In another embodiment, the membranes with a hollow fibre configuration have an external diameter of at least 100 μm. In another embodiment, the external diameter of the membranes is less than 2 mm, preferentially less than 1.5 mm, further preferentially less than 1 mm. Advantageously, the external diameter of the membranes with a hollow fibre configuration is between 100 μm and 1 mm. The internal diameter of the membranes according to the invention is preferentially less than 900 μm, even less than 850 μm. In a preferred embodiment, the external diameter of the membranes with a hollow fibre configuration is between 100 and 800 μm.

The internal diameter of the membranes with a hollow fibre configuration is generally less than 1 mm, preferentially less than 900 μm, less than 800 μm, less than 700 μm, less than 600 μm, less than 500 μm, less than 400 μm, less than 300 μm, less than 200 μm, even less than 100 μm. In one embodiment, the internal diameter of the membranes with a hollow fibre configuration according to the invention is between 1 and 100 μm. In another embodiment, the internal diameter is between 5 and 100 μm, preferentially between 7 and 100 μm, even further preferentially between 10 and 100 μm. Advantageously, the internal diameter of the membranes with a hollow fibre configuration is between 20 and 90 μm.

In reference to FIG. 2A and to FIG. 8, and according to one embodiment, the membrane contactor comprises a transfer membrane in hollow fibre configuration, which hollow fibres are dispersed in the form of a bundle of tubes. In reference to FIG. 2B, the membrane (30) is in a hollow fibre (or tube) configuration with an internal diameter and an external diameter. There is therefore an inner side (31) and an outer side (32) of the membrane, corresponding respectively to the inner side of the fibre (or tube) and the outer side of the fibre (or tube).

In one embodiment, the CO₂ supply fluid circulates inside the fibres constituting the membrane and is therefore in contact with the inner side of the membrane, while the CO₂ receiving fluid is in contact with the outer side of the membrane. In a second preferred embodiment, the CO₂ supply fluid is in contact with the outer side of the membrane and the CO₂ receiving fluid is inside the fibres and is in contact with the inner side of the membrane.

Within the framework of this application, the transfer membranes with a hollow fibre configuration are microporous membranes or non-porous membranes.

In one embodiment, the transfer membrane of the membrane contactor is a porous membrane, and comprises pores of a diameter less than 10 μm. Advantageously, the porous membrane is microporous, and presents pores with a diameter of less than 8 μm, preferentially less than 5 μm, even less than 3 μm, further preferentially less than 2 μm. In another embodiment, the microporous membrane comprises pores of a diameter of over 0.001 μm, 0.002 μm, 0.003 μm, 0.004 μm, even over 0.005 μm. Advantageously, the diameter of the pores of the membrane is then between 0.005 and 2 μm. In one embodiment, the diameter of the pores is less than 1 μm, even less than 0.5 μm, and preferentially less than 0.2 μm or even less than 0.1 μm. In another embodiment, the diameter of the pores of the membrane is between 0.005 and 0.1 μm. Also, in another embodiment, the diameter of the pores is between 0.008 and 0.09 μm, preferentially between 0.009 and 0.07 μm, even preferentially between 0.01 and 0.06 μm, and further preferentially between 0.01 and 0.05 μm. Advantageously, the diameter of the pores is around 0.01 μm, 0.02 μm, 0.03 μm, 0.04 μm or 0.05 μm.

In a preferred embodiment, the transfer membrane of the membrane contactor is a dense membrane. The diffusion of the molecules is then characterised by a phenomenon of diffusion across the membrane via the adsorption of the molecules having a strong affinity with the membrane. This embodiment presents the advantage of reducing, even preventing, clogging of the membrane of the membrane contactor with the different elements carried in the liquid medium (such as for example cellular debris) in relation to a porous membrane which presents potential adhesion areas on its structure, and thus makes it possible to maintain the same performance of gas exchanges through the membrane throughout the duration of the cultivation method.

According to one embodiment, the transfer membrane is a non-porous membrane of an internal diameter of 150 to 450 μm and an external diameter of 250 to 550 μm.

According to one embodiment, the transfer membrane is a porous membrane of an internal diameter of 150 to 250 μm and an external diameter of 250 to 350 μm and comprising pores with a diameter of 20 to 40 μm.

According to another embodiment, the transfer membrane is a dense membrane of an internal diameter of 350 to 450 μm and an external diameter of 5 to 100 mm.

Advantageously, the transfer membrane, porous or dense, particularly dense, has a thickness of less than 1,000 μm, preferentially from 100 μm to 1,000 μm.

The use of a membrane contactor according to the invention notably makes it possible to ensure all or some of the exchanges between two fluids in circulation via the presence of a transfer membrane as defined above. The membrane contactor may also be characterised by the exchange surface between the two fluids in circulation.

According to one embodiment of the invention, the membrane contactor presents an exchange surface between the two circulation fluids of at least 0.003 m²/l of CO₂ receiving fluid, preferably at least 0.006 m²/l. In parallel, the exchange surface between the two fluids in circulation is less than or equal to 0.020 m²/l per litre of CO₂ receiving fluid, preferably less than or equal to 0.017, even less than or equal to 0.015, and preferentially less than or equal to 0.012 m²/l of CO₂ receiving fluid. In another embodiment, the membrane contactor presents an exchange surface between 0.003 and 0.012 m²/l CO₂ receiving fluid, preferentially between 0.006 and 0.012 m²/I of CO₂ receiving fluid.

The materials used for the production of the transfer membranes are generally polymers, such as for example polyethylene (PE), polypropylene (PP), polysulfone (PSu), polyether sulfone (PES), polyvinylidene fluoride (PVDF), polytetrafluoroethylene (PTFE), polyimide (PI), polyamide (PA), polycarbonate (PC), polyacrylonitrile (PAN), and cellulose acetate (CA). The transfer membranes may also be manufactured from silicone or silicone derivatives. Within the framework of this application, the term silicone includes the inorganic compounds formed of a chain of silicon and oxygen atoms, and the silicon atoms of which may carry groups. Particular mention should be made of the polymers belonging to the family of siloxanes, and more specifically polydimethylsiloxane (PDMS) or compounds including polydimethylsiloxane (PDMS), notably a ternary system composed of PDMS, water and tetrahydrofuran (THF). Advantageously, the transfer membranes of this application are manufactured from silicone, preferentially PDMS, notably said membranes are thin membranes in PDMS of a thickness between 0.01 and 1.00 cm in sheet or roll. Examples of commercial transfer membranes are SSP-M823 thin silicone membranes from SSP® or Saint-Gobain PDMS. Such membrane contactors and their principles of operation are known to the person skilled in the art for other applications. They are commercially available, notably under the references Pervelys and 3M™ liqui-Cel.

The membrane contactors are thus defined as systems comprising one or more transfer membranes and whose objective is to promote the contact between two fluids in circulation, without having direct contact between the two fluids in circulation.

The use of a membrane contactor according to the invention notably makes it possible to ensure all or some of the ion or gas exchanges necessary to the optimal growth of the organisms found in the culture medium in the bioreactor.

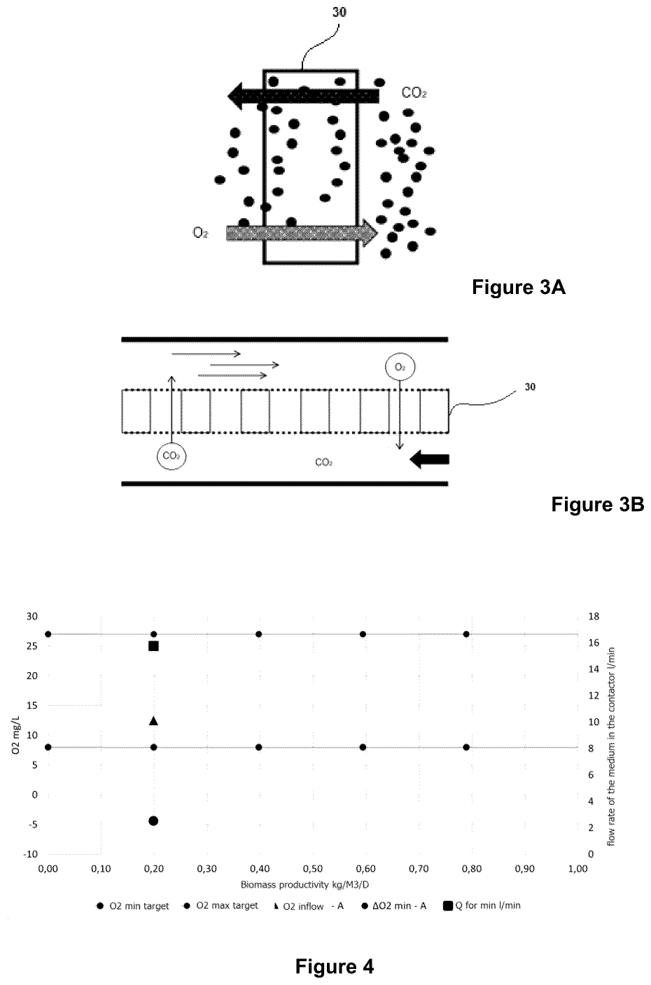

In reference to FIGS. 3A and 3B, at the transfer membrane (30) of the membrane contactor, the matter is transferred according to a pressure or concentration gradient between the two circulation fluids, the CO₂ supply fluid and the CO₂ receiving fluid. The CO₂ molecules present in the CO₂ supply fluid cross the transfer membrane of the membrane contactor, and are consumed by the microorganisms and converted into O₂ molecules in the CO₂ receiving fluid. The characteristics of the membranes and the differences in pressure and concentration between the two sides of the membrane mean the O₂ molecules cross the membrane and are found in the CO₂ supply fluid, then are eliminated from the CO₂ supply fluid by techniques well known to the person skilled in the art.

The use of the membrane contactor according to the invention notably makes it possible to ensure all or some of the ion or gas exchanges necessary to the optimal growth of the organisms found in the culture medium in the bioreactor; it is stated that said membrane contactor is a device coupled to the bioreactor and separate therefrom. Thus, the growth of the organisms takes place in the bioreactor and not in the membrane contactor, which is coupled thereto and in which the culture medium circulates.

Preferentially, the membrane contactor presents dimensions less than those of the bioreactor to which it is coupled. The coupling of a membrane contactor to a bioreactor presents several advantages, notably the improvement of the gas exchanges. Indeed, upon use of a membrane contactor, the CO₂ is supplied by permeation and is therefore completely dissolved and distributed homogenously in the medium, while a supply of CO₂ by bubbling leads to a transfer of gas only at the surface of the bubble when it passes into the medium.

Additionally, unlike CO₂ bubbling, upon use of a membrane contactor there is no desorption of CO₂ or limitation of the gas/liquid exchange surface due to the effect of coalescence of the bubbles together.

Lastly, the use of a membrane contactor makes it possible to improve the ion and gas exchanges, and consequently the consumption of CO₂ in the medium, owing to the circulation of the culture medium. This circulation is enabled by the rotation of the blades in the bioreactor or the pumping of the culture medium from the bioreactor to the membrane contactor. The culture medium is then reinjected from the membrane contactor to the bioreactor.

In one embodiment, illustrated in FIGS. 2A and 8, the membrane contactor comprises a transfer membrane of hollow fibre configuration dispersed in the form of a bundle of fibres (or tubes). The membrane contactor is thus divided into two spaces where the CO₂ supply fluid and the CO₂ receiving fluid circulate, the two spaces being separated by the transfer membrane, in this case the membrane of hollow fibre configuration.

In one embodiment, illustrated in FIG. 2A, the CO₂ supply fluid circulates in a first space (311) corresponding to the inside of the hollow fibres, with the fluid circulating according to a flow between the inlet (20) or (21) and the outlet (20) or (21) of the bundle of hollow fibres. According to this embodiment, the CO₂ receiving fluid circulates in a second space (321) bounded by the watertight walls of the membrane contactor, with the fluid circulating according to a flow between the inlet (22) or (23) and the outlet (22) or (23) of the membrane contactor.

In a second preferred embodiment, illustrated in FIG. 8, the membrane contactor comprises a transfer membrane of hollow fibre configuration dispersed in the form of a bundle of fibres (or tubes). According to this preferred embodiment, the CO₂ supply fluid circulates in a first space (321) bounded by the watertight walls of the membrane contactor, with the fluid circulating according to a flow applied between the inlet (22) or (23) and the outlet (22) or (23) of the membrane contactor. In parallel, the CO₂ receiving fluid is found in a second space (311) bounded by the membrane (inside the hollow fibres) and circulates according to a flow between the inlet (20) or (21) and the outlet (20) or (21) of the bundle of hollow fibres.

The layout of this embodiment presents the advantage of limiting the phenomenon of clogging that can arise when the CO₂ receiving fluid circulates outside the hollow fibres and the CO₂ supply fluid circulates inside the fibres, notably when the CO₂ receiving fluid is the culture medium and the CO₂ supply fluid is the gaseous CO₂.

Preferentially, the membrane contactor is placed outside the bioreactor. The membrane contactor may also be placed inside the bioreactor. If necessary, the membrane contactor may be placed in a watertight box, which can then be placed inside or outside the bioreactor.

The membrane contactors enable an effective and homogenous supply of CO₂ to all microorganisms in the cultivation methods thanks to the phenomenon of diffusion across the transfer membranes. These create a physical separation between the circulation fluid, notably between a gas phase of CO₂ supply and a liquid culture medium CO₂ receiver, allowing only some molecules to cross the membrane.

In one embodiment, the gas molecule pressure or concentration gradient results from overpressure of the CO₂ concentration. The person skilled in the art will know to determine the CO₂ flow at the entrance of the membrane contactor to be applied in order to create this overpressure, notably according to the bioreactor and the quantity of biomass desired, without this affecting the cultivation of the microorganisms.

In another embodiment, the CO₂ is injected at a regular flow according to the consumption of CO₂ necessary to the growth of the microorganisms. The CO₂ consumed is then transformed into O₂ molecules which diffuse across the transfer membrane and lead to an increase in the pressure in the gas phase of the bioreactor. An O₂ concentration threshold value may be determined by the operator in order to schedule the release of O₂ molecules to the exterior of the membrane contactor and/or the bioreactor via the air outlet.

In another embodiment, a CO₂ concentration threshold value may be determined by the operator in order to schedule the release of the CO₂ supply fluid to the exterior of the membrane contactor only when the CO₂ concentration of said supply fluid falls below said threshold value in order to enable replacement of the CO₂ supply fluid source with a CO₂ supply fluid source presenting a CO₂ concentration greater than said threshold value. In another embodiment, the concentration gradient results from a deficit of O₂ molecules in the gas phase. The absence or low concentration of O₂ molecules may be implemented notably using a pump, preferentially a vacuum pump, which eliminates the O₂ molecules from the membrane contactor and/or from the bioreactor.

Within the framework of this application, it is understood that the membrane contactor and/or the bioreactor may include other devices and apparatus ensuring the safety of the cultivation of the microorganisms and of the operators.

The CO₂ concentration is thus regulated by at least one of the previous devices, i.e. via a pressure or concentration gradient resulting from an overpressure of CO₂ and/or O₂ in the membrane contactor, or even a low O₂ concentration in the membrane contactor.

Preferentially, the CO₂ concentration is regulated with an overpressure of CO₂ in the membrane contactor.

The pressure in the membrane contactor is monitored using a manometer and adjusted with a control valve.

It should be noted that the concentration of dissolved gas in the culture medium depends on the temperature of the culture medium. Thus, the lower the temperature, the greater the concentration of dissolved gas, and vice versa.

In one embodiment, the concentration of dissolved CO₂ in the culture medium is thus between 0 and 3,500 mg/l. In another embodiment, the concentration of dissolved CO₂ is at least 100 mg/l, even at least 200 mg/l, preferentially at least 400 mg/l, further preferentially at least 600 mg/l. In another embodiment, the concentration of dissolved CO₂ in the culture medium is less than 3,400 mg/l, even less than 3,200 mg/l, preferentially less than 3,000 mg/l. In another embodiment, the concentration of dissolved CO₂ in the culture medium is between 600 and 3,000 mg/l, preferentially between 600 and 2,500 mg/l, further preferentially between 600 and 2,000 mg/l. Advantageously, the concentration of dissolved CO₂ in the culture medium is between 600 and 1,500 mg/l, even between 800 and 1,500 mg/l. In a preferred embodiment, the concentration of dissolved CO₂ is at least 1,000 mg/l, even at least 1,200 mg/l. In a further preferred embodiment, the concentration of dissolved CO₂ is between 1,000 mg/l and 1,500 mg/l, even 1,200 mg/l and 1,500 mg/l. In this latter embodiment, the temperature of the culture medium is preferentially ambient temperature (i.e. 18° C. to 25° C.).

In parallel, the quantity of dissolved O₂ in the culture medium is maintained at a concentration of less than 35 g/m³. In one embodiment, the concentration of dissolved CO₂ in the culture medium is less than 34 g/m³, even 33 g/m³, preferentially less than 32 g/m³, even further preferentially less than 31 g/m³. In another embodiment, the quantity of dissolved O₂ in the culture medium is at least 8 g/m³. In a preferred embodiment, the concentration of dissolved O₂ in the culture medium is between 8 and 30 g/m³.

Advantageously, the CO₂ concentration in the culture medium is regulated so as to provide the necessary and sufficient quantity for the cultivation of the microorganisms, such that all the CO₂ is consumed without loss into the atmosphere. The bioreactor is equipped with a control system for this regulation.

Target values are determined and entered into the control system or each of the parameters that must be controlled. The control system measures in the culture medium the parameters that must be controlled. Notably, the control system measures the pH, the temperature and the concentration of dissolved CO₂ in the culture medium. These measurements of the parameters may be taken continuously or discontinuously.

The values of the parameters thus measured are then treated and compared to the target values. As the case may be, the control system indicates the presence of a discrepancy between the values measured and the target values. This indication entails the adjustment, manual or automatic, of the parameter concerned, for example by the addition of an acid or base solution into the culture medium, the heating or cooling of the culture medium, and/or the increase or reduction of the CO₂ supply in the culture medium.

In particular, the only gas exchanges between the reactor and the external environment take place via the membrane contactor.

According to one particular embodiment of the invention, the CO₂ injected derives from a CO₂-rich gas that should be treated in order to eliminate the CO₂, without releasing it into the atmosphere. In particular, the CO₂ is a CO₂-rich gaseous industrial effluent. In particular, the CO₂-rich gaseous industrial effluent has been pretreated so as to eliminate microparticles and/or toxic components that could affect the cultivation of the microorganisms.

It should be noted that the most influential factors for the changes of concentration:
of dissolved O₂ are the initial concentration of O₂ in the liquid and the flow of the liquid in the membrane contactor (see Example 1 on the model of a Chlorella vulgaris culture in photobioreactor);
of dissolved CO₂ in the liquid are the pressure of the CO₂ in the CO₂ supply fluid in the membrane contactor, the content of CO₂ in the CO₂ supply fluid, and the initial concentration of dissolved CO₂ in the culture medium; the temperature of the liquid, the gas flow rate, the liquid flow rate and the initial concentration of O₂ in the liquid have no significant influence on the speed of transfer of CO₂ (see Example 2 on the model of a Chlorella vulgaris culture in photobioreactor).

FIG. 3 represents the flows of CO₂ and O₂ at the transfer membrane of the membrane contactor.

In reference to FIGS. 3, at the transfer membrane (30), phenomena of exchange between the CO₂ and O₂ molecules occur. While the CO₂ diffuses from the interior to the exterior of the membranes, the O₂ molecules cross the membrane from the culture medium and are eliminated at the same time as the surplus CO₂ molecules. FIG. 3B represents an embodiment where the circulation fluids are crossed. The CO₂ supply fluid and the CO₂ receiving fluid circulate in opposite directions.

Microorganisms and cultivated microorganisms are well known to the person skilled in the art. They are notably bacteria, yeasts, or protists, and more specifically algae and microalgae. According to one preferred embodiment, the cultivated microorganisms are protists.

The term protist covers all single-celled eukaryotic microorganisms. Microalgae (Chlorophytes such as Chlorella, Senedesmus, Tetraselmis, Haematococcus; Charophytes, chrysophytes are diatoms; Nannochloropsis; Euglenophytes such as Euglena, Phacus; Rhodophytes including Galdieria, etc), single-celled fungi (Thraustochytrids such as Schizochytrium, Aurantiochytrium, etc), cyanobacteria (Anabaena, Nostoc, Microcystis, Arthrospira, Spirulina, etc) and heterotrophic flagellates (Crypthecodinium etc.) belong to the group of protists.

In one embodiment, the microorganisms according to the invention are algae and microalgae preferentially belonging to the classes of Chlorophytes, Rhodophytes, Thraustochytrids and Diatoms.

Where the microalgae are of the Amphidinium genus, they may be chosen from among the species Amphidinium acutum, Amphidinium aloxalocium, Amphidinium asymmetricum, Amphidinium bipes, Amphidinium carbunculus, Amphidinium carterae, Amphidinium carteri, Amphidinium celestinum, Amphidinium coeruleum, Amphidinium conradi, Amphidinium corallinum, Amphidinium crassum, Amphidinium cyaneoturba, Amphidinium dubium, Amphidinium elegans, Amphidinium extensum, Amphidinium flagellans, Amphidinium flexum, Amphidinium fusiforme, Amphidinium glaucum, Amphidinium globosum, Amphidinium hoefleri, Amphidinium hyalinum, Amphidinium incoloratum, Amphidinium inflatum, Amphidinium kesslitzi, Amphidinium klebsii, Amphidinium lacustre, Amphidinium lanceolatum, Amphidinium latum, Amphidinium lilloense, Amphidinium longum, Amphidinium luteum, Amphidinium machapungarum, Amphidinium macrocephalum, Amphidinium mammillatum, Amphidinium massartii, Amphidinium microcephalum, Amphidinium operculatum, Amphidinium ornithocephalum, Amphidinium ovoideum, Amphidinium ovum, Amphidinium pellucidum, Amphidinium phthartum Skuja, Amphidinium psammophilum, Amphidinium pseudogalbanum, Amphidinium purpureum, Amphidinium salinum Ruinen, Amphidinium schroederi, Amphidinium scissoides, Amphidinium sphenoides, Amphidinium steini, Amphidinium stellatum, Amphidinium subsalsum, Amphidinium tortum, Amphidinium trochodinoides, Amphidinium turbo, Amphidinium vasculum, Amphidinium vittatum, Amphidinium wislouchii.

Where the microalgae are of the Chlorella genus, they may be chosen from among the species C. acuminata, C. angustoellipsoidea, C. anitrata, C. antarctica, C. aureoviridis, C. autotrophica, C. botryoides, C. caldaria, C. candida, C. capsulata, C. chlorelloides, C. cladoniae, C. coelastroides, C. colonialis, C. communis, C. conductrix, C. conglomerata, C. desiccata, C. ellipsoidea, C. elongata, C. emersonii, C. faginea, C. fusca, C. glucotropha, C. homosphaera, C. infusionum, C. kessleri, C. koettlitzii, C. lacustris, C. lewinii, C. lichina, C. lobophora, C. luteo-viridis, C. marina, C. miniata, C. minor, C. minutissima, C. mirabilis, C. mucosa, C. mutabilis, C. nocturna, C. nordstedtii, C. oblonga, C. oocystoides, C. ovalis, C. paramecii, C. parasitica, C. parva, C. peruviana, C. photophila, C. pituita, C. pringsheimii, C. protothecoides, C. pulchelloides, C. pyrenoidosa, C. regularis, C. reisiglii, C. reniformis, C. rotunda, C. rubescens, C. rugosa, C. saccharophila, C. salina, C. simplex, C. singularis, C. sorokiniana, C. spaerckii, C. sphaerica, C. stigmatophora, C. subsphaerica, C. terricola, C. trebouxioides, C. vannielii, C. variabilis, C. viscosa, C. volutis, C. vulgaris, C. zopfingiensis. Advantageously according to the invention, the algae of the Chlorella genus may be algae chosen from among the species C. sorokiniana or C. vulgaris.

Where the microalgae are of the Euglena genus, they may be chosen, inter alia, from among the species E. viridis, E. gracilis, E. limosa, E. globosa, E. prowsei, E. polomorpha.

Where the microalgae are of the Scenedesmus genus, they may be chosen from among the species S. abundans, S. aciculatus, S. aculeolatus, S. aculeotatus, S. acuminatus, S. acutiformis, S. acutus, S. aldavei, S. alternans, S. ambuehlii, S. anhuiensis, S. anomalus, S. antennatus, S. antillarum, S. apicaudatus, S. apiculatus, S. arcuatus, S. aristatus, S. armatus, S. arthrodesmiformis, S. arvernensis, S. asymmetricus, S. bacillaris, S. baculiformis, S. bajacalifornicus, S. balatonicus, S. basiliensis, S. bernardii, S. bicaudatus, S. bicellularis, S. bidentatus, S. bijuga, S. bijugatus, S. bijugus, S. brasiliensis, S. breviaculeatus, S. brevispina, S. caribeanus, S. carinatus, S. caudato-aculeolatus, S. caudatus, S. chlorelloides, S. circumfusus, S. coalitus, S. costatogranulatus, S. crassidentatus, S. curvatus, S. decorus, S. denticulatus, S. deserticola, S. diagonalis, S. dileticus, S. dimorphus, S. disciformis, S. dispar, S. distentus, S. ecornis, S. ellipsoideus, S. ellipticus, S. falcatus, S. fenestratus, S. flavescens, S. flexuosus, S. furcosus, S. fuscus, S. fusiformis, S. gracilis, S. graevenitzii, S. grahneisii, S. granulatus, S. gujaratensis, S. gutwinskii, S. hanleyi, S. helveticus, S. heteracanthus, S. hindakii, S. hirsutus, S. hortobagyi, S. houlensis, S. huangshanensis, S. hystrix, S. incrassatulus, S. indianensis, S. indicus, S. inermis, S. insignis, S. intermedius, S. javanensis, S. jovais, S. jugalis, S. kerguelensis, S. kissii, S. komarekii, S. lefevrei, S. linearis, S. littoralis, S. longispina, S. longus, S. luna, S. lunatus, S. magnus, S. maximus, S. microspina, S. minutus, S. mirus, S. morzinensis, S. multicauda, S. multiformis, S. multispina, S. multistriatus, S. naegelii, S. nanus, S. notatus, S. nygaardii, S. oahuensis, S. obliquus, S. obtusiusculus, S. obtusus, S. olvalternus, S. oocystiformis, S. opoliensis, S. ornatus, S. ovalternus, S. pannonicus, S. papillosum, S. parisiensis, S. parvus, S. pecsensis, S. pectinatus, S. perforatus, S. planctonicus, S. plarydiscus, S. platydiscus, S. pleiomorphus, S. polessicus, S. polydenticulatus, S. polyglobulus, S. polyspinosus, S. praetervisus, S. prismaticus, S. producto-capitatus, S. protuberans, S. pseudoarmatus, S. pseudobernardii, S. pseudodenticulatus, S. pseudogranulatus, S. pseudohystrix, S. pyrus, S. quadrialatus, S. quadricauda, S. quadricaudata, S. quadricaudus, S. quadrispina, S. raciborskii, S. ralfsii, S. reginae, S. regularis, S. reniformis, S. rostrato-spinosus, S. rotundus, S. rubescens, S. scenedesmoides, S. schnepfii, S. schroeteri, S. securiformis, S. semicristatus, S. semipulcher, S. sempervirens, S. senilis, S. serrato-perforatus, S. serratus, S. serrulatus, S. setiferus, S. sihensis, S. smithii, S. soli, S. sooi, S. spicatus, S. spinoso-aculeolatus, S. spinosus, S. spinulatus, S. striatus., S. subspicatus, S. tenuispina, S. terrestris, S. tetradesmiformis, S. transilvanicus, S. tricostatus, S. tropicus, S. tschudyi, S. vacuolatus, S. variabilis, S. velitaris, S. verrucosus, S. vesiculosus, S. westii, S. weberi, S. wisconsinensis, S. wuhanensis, S. wuhuensis. Advantageously according to the invention, the algae of the Scenedesmus genus may be algae chosen from among the species S. obliquus or S. abundans.

Where the microalgae are diatoms, they may be chosen from among the following genera: Nitzschia, Navicula, Gyrosigma, Phaeodactylum, Thalassiosira, etc.

Where the microalgae are of the Nitzschia genus, they may be chosen from among the species N. abbreviata, N. abonuensis, N. abridia, N. accedens, N. accommodata, N. aciculariformis, N. acicularioides, N. acicularis (comprising all these varieties), N. acidoclinata, N. actinastroides, N. actydrophila, N. acula, N. acuminata (comprising all these varieties), N. acuta, N. adamata, N. adamatoides, N. adapta, N. adducta, N. adductoides, N. admissa, N. admissoides, N. aequalis, N. aequatorialis, N. aequora, N. aequorea, N. aerophila, N. aerophiloides, N. aestuari, N. affinis, N. africana, N. agnewii, N. agnita, N. alba, N. albicostalis, N. alexandrina, N. alicae, N. allanssonii, N. alpina, N. alpinobacillum, N. amabilis, N. ambigua, N. americana, N. amisaensis, N. amphibia, N. amphibia (comprising all these varieties), N. amphibioides, N. amphicephala, N. amphilepta, N. amphioxoides, N. amphioxys (comprising all these varieties), N. amphiplectans, N. amphiprora, N. amplectens, N. amundonii, N. anassae, N. andicola, N. angularis (comprising all these varieties), N. angulata, N. angustata (comprising all these varieties), N. angustatula, N. angustiforaminata, N. aniae, N. antarctica, N. antillarum, N. apiceconica, N. apiculata, N. archibaldii, N. arcuata, N. arcula, N. arcus, N. ardua, N. aremonica, N. arenosa, N. areolata, N. armoricana, N. asperula, N. astridiae, N. atomus, N. attenuata, N. aurantiaca, N. aurariae, N. aurica, N. auricula, N. australis, N. austriaca, N. bacata (comprising all these varieties), N. bacillariaeformis, N. bacilliformis, N. bacillum, N. balatonis, N. balcanica, N. baltica, N. barbieri (comprising all these varieties), N. barkleyi, N. barronii, N. barrowiana, N. bartholomei, N. bathurstensis, N. bavarica, N. behrei, N. bergii, N. beyeri, N. biacrula, N. bicapitata (comprising all these varieties), N. bicuneata, N. bifurcata, N. bilobata (comprising all these varieties), N. birostrata, N. bisculpta, N. bita, N. bizertensis, N. blankaartensis, N. bombiformis, N. borealis, N. bosumtwiensis, N. braarudii, N. brebissonii (comprising all these varieties), N. bremensis (comprising all these varieties), N. brevior, N. brevirostris, N. brevissima (comprising all these varieties), N. brevistriata, N. brightwellii, N. brittonii, N. brunoi, N. bryophila, N. buceros, N. bukensis, N. bulnheimiana, N. buschbeckii, N. calcicola, N. caledonensis, N. calida (comprising all these varieties), N. californica, N. campechiana, N. capensis, N. capitata, N. capitellata (comprising all these varieties), N. capuluspalae, N. carnicobarica, N. carnico-barica, N. challengeri, N. chalonii, N. chandolensis, N. chardezii, N. chasei, N. chauhanii, N. chungara, N. chutteri, N. circumsuta, N. clarissima, N. clausii, N. clementei, N. clementia, N. clevei, N. closterium (comprising all these varieties), N. coarctata, N. cocconeiformis, N. communis (comprising all these varieties), N. commutata, N. commutatoides, N. compacta, N. compressa (comprising all these varieties), N. concordia, N. confinis, N. conformata, N. confusa, N. congolensis, N. constricta (comprising all these varieties), N. consummata, N. corpulenta, N. costei, N. coutei, N. creticola, N. cucumis, N. cursoria, N. curta, N. curvata, N. curvilineata, N. curvipunctata, N. curvirostris (comprising all these varieties), N. curvula (comprising all these varieties), N. cuspidata, N. cylindriformis, N. cylindrus, N. dakariensis, N. davidsonii, N. dealpina, N. debilis, N. decipiens, N. delauneyi, N. delicatissima, N. delicatula, N. delognei, N. denticula (comprising all these varieties), N. denticuloides, N. desertorum, N. dianae, N. diaphana, N. diducta, N. didyma, N. dietrichii, N. dilatata, N. diluviana, N. dippelii, N. directa, N. diserta, N. disputata, N. dissipata (comprising all these varieties), N. dissipatoides, N. distans (comprising all these varieties), N. distantoides, N. divaricata, N. divergens, N. diversa, N. diversecostata, N. doljensis, N. draveillensis, N. droebakensis, N. dubia (comprising all these varieties), N. dubiformis, N. dubioides, N. ebroicensis, N. eglei, N. elegans, N. elegantula, N. elegens, N. elliptica, N. elongata, N. entomon, N. epiphytica, N. epiphyticoides, N. epithemiformis, N. epithemioides, N. epithemoides (comprising all these varieties), N. epsilon, N. erlandssonii, N. erosa, N. etoshensis, N. examinanda, N. eximia, N. famelica, N. fasciculata, N. febigeri, N. ferox, N. ferrazae, N. fibula-fissa, N. filiformis (comprising all these varieties), N. flexa, N. flexoides, N. fluminensis, N. fluorescens, N. fluvialis, N. fogedii, N. fonticola (comprising all these varieties), N. fonticoloides, N. fonticula, N. fontifuga, N. forfica, N. formosa, N. fossalis, N. fossilis, N. fragilariiformis, N. franconica, N. fraudulenta, N. frauenfeldii, N. frequens, N. frickei, N. frigida (comprising all these varieties), N. frustuloides, N. frustulum (comprising all these varieties), N. fruticosa, N. fundi, N. fusiformis, N. gaarderi, N. gaertnerae, N. gandersheimiensis, N. garrensis, N. gazellae, N. geitleri, N. geitlerii, N. gelida (comprising all these varieties), N. geniculata, N. gessneri, N. gieskesii, N. gigantea, N. gisela, N. glabra, N. glacialis (comprising all these varieties), N. glandiformis, N. goetzeana (comprising all these varieties), N. gotlandica, N. graciliformis, N. gracilis (comprising all these varieties), N. gracillima, N. graciloides, N. gradifera, N. graeffii, N. grana, N. grandis, N. granii (comprising all these varieties), N. granulata (comprising all these varieties), N. granulosa, N. groenlandica, N. grossestriata, N. grovei, N. gruendleri, N. grunowii, N. guadalupensis, N. guineensis, N. guttula, N. gyrosigma, N. habirshawii, N. habishawii, N. hadriatica, N. halteriformis, N. hamburgiensis, N. hantzschiana (comprising all these varieties), N. harderi, N. harrissonii, N. hassiaca, N. heidenii, N. heimii, N. hemistriata, N. heteropolica, N. heuflerania, N. heufleriana (comprising all these varieties), N. hiemalis, N. hiengheneana, N. hierosolymitana, N. hoehnkii, N. holastica, N. hollerupensis, N. holsatica, N. homburgiensis, N. hudsonii, N. hummii, N. hungarica (comprising all these varieties), N. hustedti, N. hustedtiana, N. hyalina, N. hybrida (comprising all these varieties), N. hybridaeformis, N. ignorata (comprising all these varieties), N. iltisii, N. impressa, N. improvisa, N. incerta, N. incognita, N. inconspicua, N. incrustans, N. incurva (comprising all these varieties), N. indica, N. indistincta, N. inducta, N. inflatula, N. ingenua, N. inimasta, N. innominata, N. insecta, N. insignis (comprising all these varieties), N. intermedia (comprising all these varieties), N. intermissa, N. interrupta, N. interruptestriata, N. invicta (comprising all these varieties), N. invisa, N. invisitata, N. iranica, N. irregularis, N. irremissa, N. irrepta, N. irresoluta, N. irritans, N. italica, N. janischii, N. jelineckii, N. johnmartinii, N. juba, N. jucunda, N. jugata (comprising all these varieties), N. jugiformis, N. kahlii, N. kanakarum, N. kanayae, N. kavirondoensis, N. kerguelensis, N. kimberliensis, N. kittlii, N. kittonii, N. knysnensis, N. kolaczeckii, N. kotschyi, N. kowiensis, N. krachiensis, N. krenicola, N. kuetzingiana (comprising all these varieties), N. kuetzingii, N. kuetzingioides, N. kurzeana, N. kurzii, N. kützingiana (comprising all these varieties), N. labella, N. labuensis, N. lacrima, N. lacunarum, N. lacunicola, N. lacus-karluki, N. lacustris, N. lacuum, N. laevis, N. laevissima, N. lagunae, N. lagunensis, N. lamprocampa (comprising all these varieties), N. lanceola (comprising all these varieties), N. lanceolata (comprising all these varieties), N. lancettula, N. lancettuloides, N. lange-bertalotii, N. latens, N. latestriata, N. latiuscula, N. lauenbergiana, N. lauenburgiana, N. lecointei, N. leehyi, N. legleri, N. lehyi, N. leistikowii, N. lesbia, N. lesinensis, N. lesothensis, N. leucosigma, N. levidensis (comprising all these varieties), N. liebetruthii (comprising all these varieties), N. ligowskii, N. limicola, N. limulus, N. linearis (comprising all these varieties), N. lineata, N. lineola, N. linkei, N. lionella, N. littoralis (comprising all these varieties), N. littorea, N. longa, N. longicollum, N. longirostris, N. longissima (comprising all these varieties), N. lorenziana (comprising all these varieties), N. lucisensibilis, N. lunaris, N. lunata, N. lurida, N. luzonensis, N. macaronesica, N. macedonica, N. macera, N. machardyae, N. macilenta (comprising all these varieties), N. magnacarina, N. mahihaensis, N. mahoodii, N. maillardii, N. major, N. majuscula (comprising all these varieties), N. makarovae, N. manca, N. mancoides, N. manguini, N. marginata, N. marginulata (comprising all these varieties), N. marina, N. martiana, N. maxima, N. media, N. medioconstricta, N. mediocris, N. mediterranea, N. metzeltinii, N. microcephala (comprising all these varieties), N. migrans, N. minuta, N. minutissima, N. minutula, N. miramarensis, N. miserabilis, N. mitchelliana, N. modesta, N. moissacensis (comprising all these varieties), N. mollis, N. monachorum, N. monoensis, N. montanestris, N. morosa, N. multistriata, N. nana, N. natalensis, N. natans, N. nathorsti, N. navicularis, N. navis-varingica, N. navrongensis, N. neglecta, N. nelsonii, N. neocaledonica, N. neoconstricta, N. neofrigida, N. neogena, N. neotropica, N. nereidis, N. nicobarica, N. nienhuisii, N. normannii, N. notabilis, N. nova, N. novae-guineaensis, N. novae-guineensis, N. novaehollandiae, N. nova-zealandia, N. nyassensis, N. oberheimiana, N. obesa, N. obliquecostata, N. obscura, N. obscurepunctata, N. obsidialis, N. obsoleta, N. obsoletiformis, N. obtusa (comprising all these varieties), N. obtusangula, N. oceanica, N. ocellata, N. oliffi, N. omega, N. osmophila, N. ossiformis, N. ostenfeldii, N. ovalis, N. paaschei, N. pacifica, N. palacea, N. palea (comprising all these varieties), N. paleacea, N. paleaeformis, N. paleoides, N. palustris, N. pamirensis, N. panduriformis (comprising all these varieties), N. pantocsekii, N. paradoxa (comprising all these varieties), N. parallela, N. pararostrata, N. partita, N. parvula (comprising all these varieties), N. parvuloides, N. paxillifer, N. peisonis, N. pelagica, N. pellucida, N. pennata, N. peragallii, N. perindistincta, N. perminuta, N. perpusilla (comprising all these varieties), N. perspicua, N. persuadens, N. pertica, N. perversa, N. petitiana, N. philippinarum, N. pilum, N. pinguescens, N. piscinarum, N. plana (comprising all these varieties), N. planctonica, N. plicatula, N. plioveterana, N. polaris, N. polymorpha, N. ponciensis, N. praecurta, N. praefossilis, N. praereinholdii, N. princeps, N. procera, N. prolongata (comprising all these varieties), N. prolongatoides, N. promare, N. propinqua, N. pseudepiphytica, N. pseudoamphioxoides, N. pseudoamphioxys, N. pseudoamphyoxys, N. pseudoatomus, N. pseudobacata, N. pseudocapitata, N. pseudocarinata, N. pseudocommunis, N. pseudocylindrica, N. pseudodelicatissima, N. pseudofonticola, N. pseudohungarica, N. pseudohybrida, N. pseudonana, N. pseudoseriata, N. pseudosigma, N. pseudosinuata, N. pseudostagnorum, N. pubens, N. pulcherrima, N. pumila, N. punctata (comprising all these varieties), N. pungens (comprising all these varieties), N. pungiformis, N. pura, N. puriformis, N. pusilla (comprising all these varieties), N. putrida, N. quadrangula, N. quickiana, N. rabenhorstii, N. radicula (comprising all these varieties), N. rautenbachiae, N. recta (comprising all these varieties), N. rectiformis, N. rectilonga, N. rectirobusta, N. rectissima, N. regula, N. reimeri, N. reimerii, N. reimersenii, N. retusa, N. reversa, N. rhombica, N. rhombiformis, N. rhopalodioides, N. richterae, N. rigida (comprising all these varieties), N. ritscheri, N. robusta, N. rochensis, N. rolandii, N. romana, N. romanoides, N. romanowiana, N. rorida, N. rosenstockii, N. rostellata, N. rostrata, N. ruda, N. rugosa, N. rupestris, N. rusingae, N. ruttneri, N. salinarum, N. salinicola, N. salpaespinosae, N. salvadoriana, N. sansimoni, N. sarcophagum, N. scabra, N. scalaris, N. scaligera, N. scalpelliformis, N. schoenfeldii, N. schwabei, N. schweikertii, N. scutellum, N. sellingii, N. semicostata, N. semirobusta, N. separanda, N. seriata (comprising all these varieties), N. serpenticola, N. serpentiraphe, N. serrata, N. sibula (comprising all these varieties), N. sigma (comprising all these varieties), N. sigmaformis, N. sigmatella, N. sigmoidea (comprising all these varieties), N. silica, N. silicula (comprising all these varieties), N. siliqua, N. similis, N. simplex, N. simpliciformis, N. sinensis, N. sinuata (comprising all these varieties), N. smithii, N. sociabilis, N. socialis (comprising all these varieties), N. solgensis, N. solida, N. solita, N. soratensis, N. sp., N. spathulata (comprising all these varieties), N. speciosa, N. spectabilis (comprising all these varieties), N. sphaerophora, N. spiculoides, N. spiculum, N. spinarum, N. spinifera, N. stagnorum, N. steenbergensis, N. stellata, N. steynii, N. stimulus, N. stoliczkiana, N. stompsii (comprising all these varieties), N. strelnikovae, N. stricta, N. strigillata, N. striolata, N. subaccommodata, N. subacicularis, N. subacuta, N. subamphioxioides, N. subapiculata, N. subbacata, N. subcapitata, N. subcapitellata, N. subcohaerens (comprising all these varieties), N. subcommunis, N. subconstricta, N. subcurvata, N. subdenticula, N. subfalcata, N. subfraudulenta, N. subfrequens, N. subfrustulum, N. subgraciloides, N. subinflata, N. subinvicta, N. sublaevis, N. sublanceolata, N. sublica, N. sublinearis, N. sublongirostris, N. submarina, N. submediocris, N. subodiosa, N. subpacifica, N. subpunctata, N. subromana, N. subrostrata, N. subrostratoides, N. subrostroides, N. subsalsa, N. subtilioides, N. subtilis (comprising all these varieties), N. subtubicola, N. subvitrea, N. suchlandtii, N. sulcata, N. sundaensis, N. supralitorea, N. tabellaria, N. taenia, N. taeniiformis, N. tantata, N. tarda, N. taylorii, N. temperei, N. tenella, N. tenerifa, N. tenuiarcuata, N. tenuirostris, N. tenuis (comprising all these varieties), N. tenuissima, N. tergestina, N. terrestris, N. terricola, N. thermalis (comprising all these varieties), N. thermaloides, N. tibetana, N. tirstrupensis, N. tonoensis, N. towutensis, N. translucida, N. tropica, N. tryblionella (comprising all these varieties), N. tsarenkoi, N. tubicola, N. tumida, N. turgidula, N. turgiduloides, N. umaoiensis, N. umbilicata, N. umbonata, N. vacillata, N. vacua, N. valdecostata, N. valdestriata, N. valens, N. valga, N. valida (comprising all these varieties), N. vanheurckii, N. vanoyei, N. vasta, N. ventricosa, N. vermicularioides, N. vermicularis (comprising all these varieties), N. vermicularoides, N. vexans, N. victoriae, N. vidovichii, N. vildaryana, N. villarealii, N. virgata, N. visurgis, N. vitrea (comprising all these varieties), N. vivax (comprising all these varieties), N. vixnegligenda, N. vonhauseniae, N. vulga, N. weaveri, N. weissflogii, N. westii, N. williamsiii, N. wipplingeri, N. witkowskii, N. wodensis, N. woltereckii, N. woltereckoides, N. wuellerstorfii, N. wunsamiae, N. yunchengensis, N. zebuana, N. zululandica.

Advantageously according to the invention, the algae of the Nitzschia genus may be algae chosen from among the species N. sp.

Where the microalgae are of the Haematococcus genus, they may be chosen from among the species: H. buetschlii, H. capensis, H. carocellus, H. droebakensis, H. grevilei, H. insignis, H. lacustris, H. murorum, H. pluvialis, H. salinus, H. sanguineis, H. thermalis, H. zimbabwiensis.

Where the microalgae are of the Tetraselmis genus, they may be chosen from among the species: T. alacris, T. apiculata, T. arnoldii, T. ascus, T. astigmatica, T. bichlora, T. bilobata, T. bolosiana, T. chui, T. contracta, T. convolutae, T. cordiformis, T. desikacharyi, T. elliptica, T. fontiana, T. gracilis, T. hazenii, T. helgolandica, T. impellucida, T. incisa, T. inconspicua, T. indica, T. levis, T. maculata, T. marina, T. mediterranea, T. micropapillata, T. rubens, T. striata, T. subcordiformis, T. suecica, T. tetrabrachia, T. tetrathele, T. verrucosa, T. viridis, T. wettsteinii.

According to one particular embodiment of the invention, the protists are chosen from among the genera Chlorella, Galdieria, Euglena, cyanobacteria and diatoms.

The microorganisms may be cultivated in the absence or presence of a carbon source, and/or in the absence or presence of light. The method according to the invention is thus adapted to the cultivation of microorganisms in heterotrophic, autotrophic or mixotrophic mode, In a preferred embodiment, the microorganisms are microalgae cultivated according to an autotrophic or mixotrophic mode. Advantageously, the microorganisms cultivated according to the invention are autotrophic microalgae.

The method of cultivation of microorganisms comprises the cultivation of said microorganisms in a bioreactor coupled to the membrane contactor according to the invention, and the recovery of the biomass produced by the microorganisms. The biomass produced may be used for the preparation of pharmaceutical, cosmetic, nutraceutical or food compounds.

The invention thus also concerns the use of a membrane contactor to regulate the flows of CO₂ and O₂ in a method of cultivation of microorganisms wherein the CO₂ is a source of carbon in the culture medium. The membrane contactor may be placed inside or outside the bioreactor. It is understood that the membrane contactor may be placed in a box, which may be inside or outside the bioreactor. Preferentially, the membrane contactor is placed inside the bioreactor. The microorganisms and other cultivation means were described previously.

The invention also concerns a method of decarbonation of a CO₂-rich gas which comprises the injection of said gas in a bioreactor, said bioreactor comprising a culture medium and microorganisms capable of growing in said culture medium with the CO₂ as sole carbon source and CO₂ supply means, characterised in that the CO₂ supply means comprise a membrane contactor to regulate the flows of CO₂ and O₂ in the culture medium, the flow of CO₂ being directed to the culture medium, and the flow of O₂ being directed to the exterior of the culture medium.

Advantageously, the concentration of CO₂ in the culture medium is regulated so as to provide the necessary and sufficient quantity for the cultivation of the microorganisms, such that all the CO₂ is consumed without loss into the atmosphere.

In particular, in the method of decarbonation according to the invention, the only gas exchanges between the reactor and the outside environment take place via the membrane contactor. The reactor is advantageously closed.

Lastly, the invention concerns a fermentation device for microorganisms, said microorganisms being microalgae according to the invention. The fermentation device comprises:
(a) a fermentation tank or bioreactor, and
(b) a CO₂ supply means, comprising a membrane contactor according to the invention enabling regulation of the flows of CO₂ and O₂, the flow of CO₂ being directed to the culture medium and the flow of O₂ being directed to the exterior of the culture medium.

In reference to FIG. 1, the device comprises a membrane contactor (2) coupled to the bioreactor (1). The circulation of the CO₂ receiving fluid (the flow of the culture medium) contained in the bioreactor (3) and (4) is assured thanks to a pump (5) arranged between the bioreactor and the membrane contactor. Measuring sensors or probes (10) and (11) can be added to the device. These are notably sensors measuring CO₂ and O₂.

The membrane contactor is moreover linked to a CO₂ source which induces a flow of CO₂ (CO₂ supply fluid) in the membrane contactor (6) and (7). The device according to the invention may also include a vacuum pump (8) in order to create a flow of CO₂. The device may furthermore include a buffer tank (9) enabling the storage of CO₂. Preferably, the buffer tank is placed between the CO₂ supply source and the membrane contactor.

In reference to FIG. 8, the membrane contactor is connected to the source of the carbon dioxide (CO₂) at the CO₂ supply fluid (22) inlet. The CO₂ supply fluid leaves the membrane contactor (23) and returns according to the closed circuit in the CO₂ supply tank (9). In parallel, the CO₂ receiving fluid circulates in the membrane contactor from the inlet (20) connected to the bioreactor (1) to the outlet (21), which outlet is also connected to the bioreactor (1). There are therefore two circulation flows within the device: a flow of circulation of the CO₂ supply fluid (6) and (7), and a flow of circulation of the CO₂ receiving fluid (3) and (4). These two flows circulate according to a closed circuit and pass into the membrane contactor where the exchanges of matter occur between the two fluids.

According to the invention, the membrane contactor comprises a transfer membrane, preferentially a transfer membrane with a hollow fibre configuration. In this embodiment, the hollow fibre transfer membrane is chosen from among microporous membranes and dense membranes. Preferentially the transfer membrane with a hollow fibre configuration is a dense membrane.

In the fermentation device, the membrane contactor may be placed inside or outside the reactor. It is understood that the membrane contactor may be placed in a box, itself placed inside or outside the bioreactor. In a preferred embodiment, the membrane contactor is placed outside the reactor.

According to one particular embodiment, the reactor is closed, isolated from the outside, with the only gas exchanges with the exterior taking place via the membrane contactor with a supply of CO₂ and an evacuation of O₂.

### EXAMPLES

### Materials and Methods

### Determination of Cell Growth

### Optical Density (OD)

OD measurements are taken in duplicate at 750 nm with a spectrophotometer (7315, JENWAY). The daily productivity is calculated from the OD and from a correlation coefficient (CC) specific to the microorganism cultivated according to the following formula, particularly when the biomass concentration is too low to have a precise dry mass value.

$$Productivity ( OD ) ( g . {L}^{- 1} . {day}^{- 1} ) = \frac{( OD ( day D ) - OD ( day D - 1 ) ) \times C C}{time ( day D ) - time ( day D - 1 )} \times 24$$

### Dry Mass (DM)

DM measurements are taken in duplicate using a vacuum filtration collector. For that purpose, a sample is placed on an AP40 fibreglass prefilter (pores of 0.7 μm, Merck Millipore). The filtered dry matter is then weighed after incubation for 24 hours at 100° C. The final DM value is calculated according to the following formula.

$$DM ( g . {L}^{- 1} ) = \frac{final mass - initial mass}{volume of the sample}$$

The following formula is used to calculate the daily productivity from the DM.

$$Productivity ( DM ) ( g . {L}^{- 1} . {day}^{- 1} ) = \frac{( DM ( day D ) - DM ( day D - 1 ) )}{time ( day D ) - time ( day D - 1 )} \times 24$$

### Example 1: Variation in the Quantity of Dioxygen (O₂)

Aim: To eliminate the dissolved O₂ according to the geometric and physicochemical characteristics of the reactor by varying the flow rate of the medium in the contactor.

Experimental conditions: cultivation of a biomass of Chlorella vulgaris with a photobioreactor in which the light-emitting surface remains constant (250 m²). The culture medium used is described in Table 1 below.

TABLE 1

| Nutrients | Concentration g/l |
|---|---|
| KNO3 | 8.888 |
| NaH2PO4 | 1.238709677 |
| CaCl2, 2H2O | 0.04672 |
| MgSO4, 7H2O | 1.312 |
| Ferric EDTA | 0.577 |

The quantity of photons emitted by the light surfaces vary from 1,050 to 0 μmol photons/m²/s. There is no unlit zone in the reactor (fd=0).

It is possible to determine the speed that must be applied to the flow of the culture medium over the transfer membranes according to the quantity of O₂ present in the medium and of that produced by the microalgae according to the configurations of the photobioreactor.

The concentration of dissolved dioxygen must be controlled between 8 g/m³ and 30 g/m³. Indeed, it has been found that a concentration of dissolved oxygen below 8 g/m³ may have a negative effect on the growth of the microalgae, while a solubility of O₂ greater than 30 g/m³ may induce a loss of productivity of the biomass on Chlorella vulgaris.

The quantity of O₂ produced is modelled via the production of the biomass with the following method:

$$P O 2 = \frac{Y O 2}{x} * VP ,$$

where YO₂/x is equal to 1.5 kgO₂/kg biomass produced and VP is the volume productivity.

Consequently, the flow of the medium in the membrane contactor depends on ΔO₂ corresponding to the following formula:

$${ΔO}_{2} ( mg / I ) = - 0.79 - 0.66 * initial {O}_{2} concentration ( mg / I ) + 0.29 * speed of flow of the medium ( I / min )$$

Case A: the system is sized to produce 0.2 kg/m³/day and the production of O₂ will remain between the desirable minimum and maximum limits of dissolved O₂. In order to reach the minimum limit, a flow rate of 15.72 l/min O₂ is necessary (FIG. 4).

The production of O₂ remains reasonable because it remains within the tolerable limits for good photosynthesis. It is nevertheless possible to stay as close as possible to the minimum limit by adjusting the flow rate. Secondly, if the productivity increases, the production of O₂ will be harmful for the microalgae because it is above those limits. The flow in the transfer membranes must then be adjusted so as to reach the maximum desirable limit of dissolved O₂.

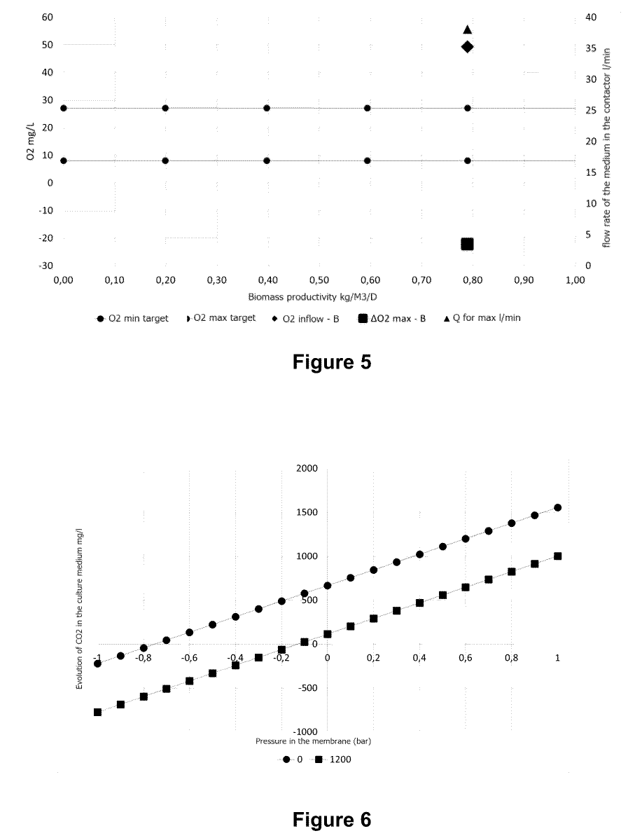

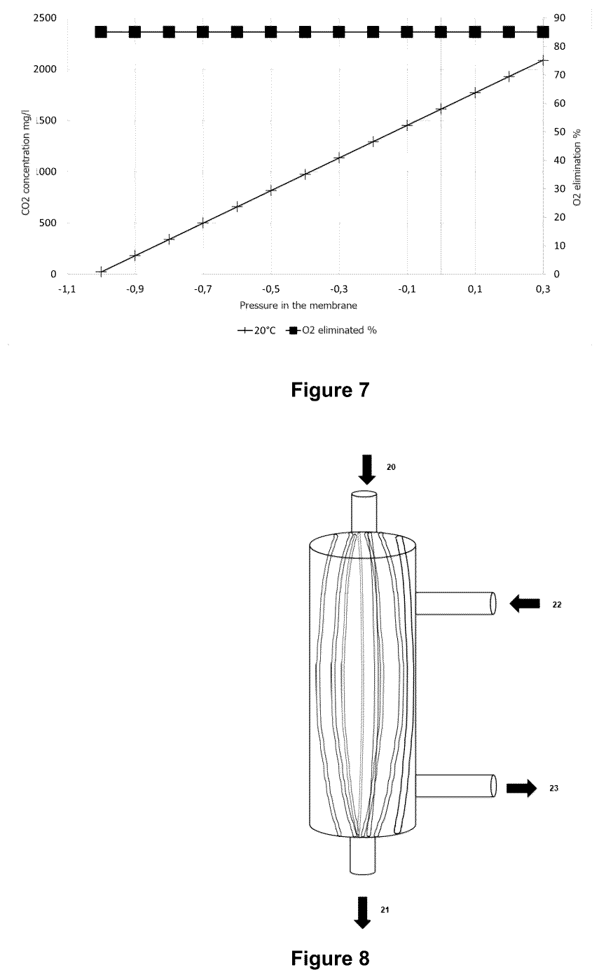

Case B: the system is defined to produce 0.79 kg/m³/day; the production of O₂ will be above the maximum limit. The O₂ concentration must therefore be reduced by 22.25 mg/l, through a flow rate in the transfer membranes of around 38.07 l/min (FIG. 5).

Table 1bis summarises the different parameters for systems A and B.

TABLE 1BIS

| | Productivity A | Productivity B |
|---|---|---|
| Productivity | 0.20 | 0.79 |
| (gram/litre/Day) | | |
| Min Limit O₂ | 8 | 8 |
| (milligram/litre) | | |
| Max Limit O₂ | 27 | 27 |
| (milligram/litre) | | |
| Inlet m O₂ | 12 | 49 |
| (milligram/litre/hour) | | |
| Min ΔO₂ (milligram/litre) | −4 | −41 |
| Max ΔO₂ | 15 | −22 |
| (milligram/litre) | | |
| Flow rate (Q) for O₂ Min | 16 | undetermined |
| Limit (litre/minute) | | |
| Flow rate (Q) for O₂ | 0 | 38 |
| Max Limit (litre/minute) | | |

### Example 2: Variation in the Quantity of Carbon Dioxide (CO₂)

It is possible to increase or reduce the quantity of dissolved CO₂ according to the quantity initially present in the culture medium by varying the conditions of pressure on the transfer membrane of the contactor. The variation in the quantity of CO₂ can thus be defined according to the following equation:

$${ΔCO}_{2} ( mg / I ) = 664.96 - 0.46 * initial {CO}_{2} concentration ( mg / I ) + 888.3 * pressure ( bar )$$

If the concentration of the culture medium comprises a concentration of 100 mg/l CO₂ and a constant concentration of dissolved CO₂ of 1,200 mg/l is required, the CO₂ delta of 1,100 mg/l can be addressed with a pressure on the transfer membrane of 0.542 bar (FIG. 6). This maintenance of CO₂ must be corrected over time according to the quantity of photon-emitting surfaces sent into the tank.

Various parameters can be adjusted to calculate productivities in a photobioreactor. They are taken into account in the below model of the surface output of a photobioreactor.

$$\begin{matrix}〈 {r}_{X}^{′} 〉 ≅ ( 1 - {f}_{d} ) {ρ}_{M} {\overline{ϕ}}^{′} \frac{2 α}{1 + α} {a}_{light} \frac{K}{( \frac{\overline{n} + 2}{\overline{n} + 1} )} ln [ 1 + \frac{( \frac{\overline{n} + 2}{\overline{n} + 1} ) {\overline{q}}_{0}}{K} ] \\ = ( 1 - {f}_{d} ) {ρ}_{M} {\overline{ϕ}}^{′} {〈 A 〉}_{max} \frac{K}{( \frac{\overline{n} + 2}{\overline{n} + 1} )} ln [ 1 + \frac{( \frac{\overline{n} + 2}{\overline{n} + 1} ) {\overline{q}}_{0}}{K} ]\end{matrix}$$

Where:
(r′x)ₘₐₓ: maximum surface output of the photobioreactor
K: half-saturation constant of the photosynthesis
alight: corresponds to the ratio between the lit surface and the real volume of the photobioreactor (S_{Light}/Vr)
Vr: Real volume
ň: average degree of collimation of the incidental radiation
fd: dark fraction or ratio of unlit volume to the real volume of the photobioreactor
A: volume density of the radiating power absorbed in μmolhv·m−3·s−1
ρM: maximum energy output from conversion of light energy into physicochemical energy
Qn: average photon flow density on the surface of the photobioreactor
h: Planck's constant
v: frequency
Sx: surface production
Px continuous: volume production
Corrected output=Px with a corrective factor of 20%

The production of the photobioreactor will depend mainly on the following elements:
The dark fraction (fd)
The surface production (Sx) due to the capture of the surface photon flow
The volume production (Px) according to the surface photon flow in relation to the total volume. In this case, it is preferable to reduce the unlit fraction of the reactor as much as possible and to increase the surface receiving the flow of photons.

The quantity of CO₂ to be supplied in order to achieve the target and the surplus for the growth of microalgae is calculated according to the following formula:

$${ΔCO}_{2} * {V}_{r e a c t o r} + ( biomass productivity * {V}_{reactor} * Assimilation kg CO 2 per kgbiomass )$$

The parameters of Example 2 are summarised in Table 2 below.

TABLE 2
Case of reactor geometry

| | | |
|---|---|---|
| Light surface (m²) | 25 | 25 |
| Qn (μmolhv/m²/s) | 800.00 | 200.00 |
| K (μmolhv/kgbiomass/s) | 30000.00 | 30000.00 |
| Vr (m³) | 1.00 | 1.00 |
| alight (m⁻¹) | 25.00 | 25.00 |
| Fd (%) | 0.00 | 0.00 |
| Sx (kilogram/metre²/Day) | 0.03 | 0.01 |
| Px continuous (kilogram/m³/Day) | 0.66 | 0.17 |
| Corrected output (kilogram/m³/ | 0.79 | 0.20 |
| Day) | | |
| Assimilation (kg_{CO2}/kg_{biomass}) | 2.059 | 2.059 |
| kg_{CO2}/biomass/Day | 1.62552958 | 0.41041054 |
| Initial CO₂ concentration (mg/l) | 100 | 100 |
| Target CO₂ concentration (mg/l) | 1200 | 1200 |
| ΔCO₂ (mg/l) | 1100 | 1100 |
| V (L) | 1000 | 1000 |
| Pressure necessary for target CO₂ | 0.542 | 0.542 |
| (bar) | | |
| Quantity of CO₂ to be supplied to | 2.726 | 1.510 |
| reach the target and the surplus for | | |
| the growth of the microalgae | | |
| (kg_{CO2}/Day) | | |

### Example 3: Quantity of CO₂ to be Injected into the Culture Medium According to Temperature and Pressure

Aim: To eliminate the O₂ produced by the photosynthesis of the microalgae via the injection of concentrated CO₂ which will take the role of a carrier gas.

Experimental conditions: Cultivation of a biomass of Chlorella vulgaris with the culture medium described in Example 1 (Table 1) in a photobioreactor with 25 m² light surface and 1,050 μmol photons/m²/s surface emission. The biomass production is estimated at 1.03 kg/m³/D, which is a CO₂ consumption of 2.12 kgCO₂/kgBiomass/m³/day.

The CO2 flow is adjusted so as to eliminate at least 85% of initial O₂ produced. The parameters are then verified via manometers and liquid and gas flowmeters.

The O₂ gas pressure of the incoming gas is maintained at below the O₂ gas pressure of the culture medium (PO₂ incoming gas <PO₂ culture medium). The CO₂ gas pressure of the incoming gas is maintained at above the CO₂ gas pressure of the culture medium (PCO₂ incoming gas >PCO₂ culture medium).

The quantity of CO₂ injected depends on the temperature of the culture medium, the initial concentration of the culture medium, the pressure allowed in the transfer membrane carrying the CO₂, and the difference in CO₂ concentration between the initial and target CO₂ concentrations.

When the culture medium is saturated with CO₂, an additional quantity of CO₂ must be added in order to serve as carrier for the O₂, but also in anticipation of the consumption of CO₂ by the biomass. This method makes it possible to maintain a maximum availability of CO₂ according to the strain in correlation with its consumption.

Table 3 adopts the different parameters enabling calculation of ΔCO₂ and the CO₂ pressure that must be reached in order to obtain a given saturation of dissolved CO₂, according to the temperature of the culture medium. These parameters thus also enable calculation of the quantity of CO₂ to be added for the growth of the microalgae and elimination of the O₂.

TABLE 3

| | | |
|---|---|---|
| Temperature | ° Celsius | 20 |
| | ° Fahrenheit | 68 |
| CO₂ Density at | kg/m³ | 1.82818281 |
| Initial CO₂ concentration | mg/L | 300 |
| Target CO₂ concentration | mg/L | 1200 |
| ΔCO₂ | mg | 900 |
| V | L | 1000 |
| Quantity of CO₂ to be | kg | 0.9 |
| supplied for the target | | |
| CO₂ pressure necessary for | Bar | −0.263298 |
| the target | | |

Regulate Q to adjust % O₂ eliminated

| | | |
|---|---|---|
| Quantity of CO₂ to be supplied to reach | kg/Day | 3.02485775 |
| the target and the surplus for the growth of | | |
| the microalgae | | |

On the same principle as in Example 2, the parameters of Example 3 are summarised in Table 4 below.

TABLE 4
Case of reactor geometry

| | |
|---|---|
| Light surface (m²) | 25 |
| Qn (μmolhv/m²/s) | 1050 |
| K (μmolhv/kgbiomass/s) | 30000 |
| Vr (m3) | 1.00 |
| A light (m−1) | 25 |
| Fd (%) | 0 |
| Sx (kilogram/m²/Day) | 0.03 |
| Px continuous | 0.86 |
| (kilogram/m³/Day) | |
| Corrected output | 1.03 |
| (kilogram/m³/Day) | |
| Assimilation | 2.059 |
| (kgCO2/kgbiomass) | |
| kgCO2/biomass/Day | 2.12485775 |

### Example 4: Cultivation of Chlorella sorokiniana with and without the Use of a Membrane Contactor for the Supply of CO₂

Cultures of Chlorella sorokiniana are launched in two 5-litre STR bioreactors (Pierre Guérin, working volume 3 L). The system comprises a peristaltic pump (MasterFlex® model 7518-00, Cole Parmer). The two reactors only differ by the means used to supply the CO₂ (the quantity of CO₂ supplied remaining the same). One is equipped with a membrane contactor according to the invention and a bubbling means. The membrane contactor is a structure of 200 mm by 550 mm comprising a transfer membrane with a configuration of dense gas-permeable silicone fibres of an internal diameter of around 200 μm and external of around 300 μm, wherein the culture medium is circulated using to a WG600S peristaltic pump, 400 rpm.

A diagrammatic representation of the membrane contactor used is presented in FIG. 8.

The second (control) bioreactor is not equipped with a membrane contactor but a CO₂ bubbling system.

The cultivation is maintained for 7 days under light of intensity of 500 μmol·m⁻²·s⁻¹ (white, 4000K).

The culture medium used is that described in Example 1 (Table 1), and each bioreactor is inoculated at a volume of 1.5% with a biomass incubated from cryotubes (INOVA), target OD 0.05.

The cultivation parameters are maintained as follows:
pH: between 7 and 8:
temperature: 30° C.;
agitation: 200 rpm;
pressure on the transfer membrane of the contactor: 0.1 bar, using a control valve and a manometer;

In the first 48 hours, the culture is supplied with air (1 m³.h⁻¹) then with CO₂ (0.05 l/min). Once to 3 times per day, a sample of 15 ml is taken for OD and DM analyses. The temperature, pH and CO₂ and O₂ pressure parameters are monitored.

After a few days, a difference in colour between the biomasses of each bioreactor is visible to the naked eye. After 3 days of cultivation, in the case of the bioreactor according to the invention, a CO₂ pressure is applied to the transfer membrane of the contactor using a stop valve, firstly of 0.05 bar, then 0.20 bar, then 0.225 bar, then 0.25 bar. The O₂ pressure then tends to reduce within the reactor. The target CO₂ pressure applied has been determined according to the method described in Example 3 above, and enables the promotion of gas exchanges (O₂ and CO₂) across the transfer membrane.

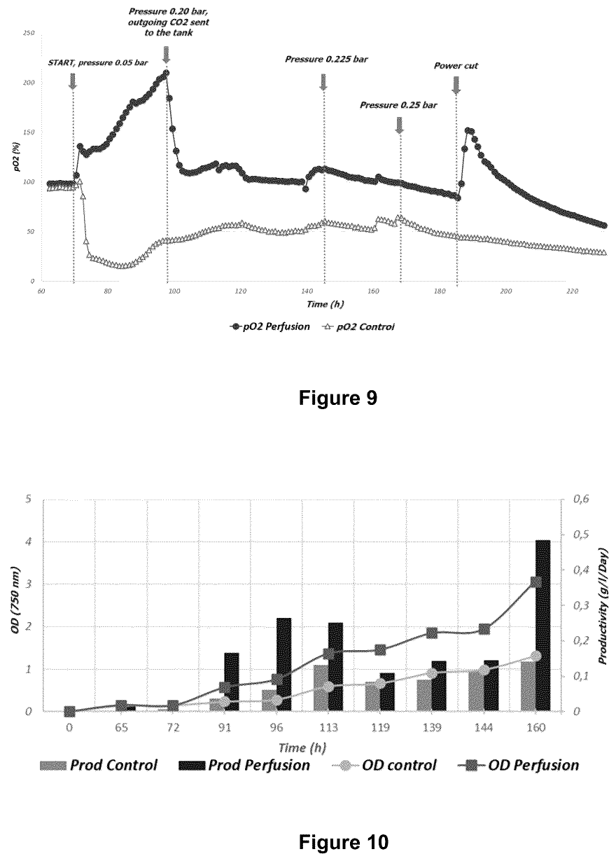

A graph showing the evolution over time of the O₂ pressure in the culture medium is presented in FIG. 9. It should be noted that a power cut occurred on day 8. Consequently, in addition to the temporary absence of light and agitation, the pump enabling circulation of the medium in the contactor was interrupted. This is visible on the graph: on day 8 a peak O₂ pressure is present.

The growth of Chlorella sorokiniana is visible in the two bioreactors. The results of OD and daily productivity (based on the OD) are presented in FIG. 10. However, from day 4 the daily production (based on the OD) is considerably higher for the culture in the bioreactor according to the invention in comparison to the control. This trend is maintained over the whole duration of the cultivation. Considering that the only difference between the two bioreactors is the use or absence of use of a membrane contactor in compliance with the invention, these differences in productivity indicate the improved supply of CO₂ and the effective elimination of O₂.

It should be noted that, during the cultivation, there was only moderate clogging of the membrane contactor and that the medium therefore could be stirred uniformly. Furthermore, the appearance of foam seems to have been limited in the case of the bioreactor equipped with a membrane contactor according to the invention.

The maximum theoretical productivities are achieved after 7 days for the 2 cultures (0.48/g/L/day for the invention and 0.14 for the control).

1. Method of cultivation of a microorganism in a culture medium comprising CO₂ as carbon source and comprising a step of cultivation of the microorganism in a bioreactor comprising CO₂ supply means, characterised in that the CO₂ supply means comprise a membrane contactor for regulating the flows of CO₂ and O₂ in the culture medium, and wherein the flow of CO₂ is directed to the culture medium and the flow of O₂ is directed to the exterior of the culture medium through the transfer membrane of the membrane contactor.

2. Method of cultivation according to claim 1, characterised in that the membrane contactor comprises at least two fluid circulation circuits: a first circulation circuit of a CO₂ supply fluid and a second circulation circuit of a CO₂ receiving fluid, and a transfer membrane separating said circulation circuits.

3. Method according to claim 1 or 2, characterised in that the membrane contactor comprises a transfer membrane with a hollow fibre configuration.

4. Method according to one of claims 1 to 3, characterised in that the membrane contactor comprises a microporous transfer membrane or a non-porous transfer membrane, preferentially a non-porous transfer membrane.

5. Method according to one of claim 3 or 4, characterised in that the external diameter of the membrane(s) is between 100 and 800 μm.

6. Method according to one of claims 1 to 5, characterised in that the membrane contactor comprises a microporous transfer membrane whereby the pores have a diameter between 0.01 and 0.5 μm.

7. Method according to one of claims 2 to 6, characterised in that the CO₂ supply fluid circulates outside the hollow fibres.

8. Method according to one of claims 2 to 7, characterised in that the CO₂ receiving fluid circulates inside the hollow fibres.

9. Method according to one of claims 1 to 8, characterised in that the concentration of CO₂ in the culture medium is at least 1,000 mg/l.

10. Method according to one of claims 1 to 9, characterised in that the quantity of O₂ in the reactor is less than 35 g/m³, preferentially between 8 g/m³ and 33 g/m³.

11. Method according to one of claims 1 to 10, characterised in that the concentration of CO₂ in the culture medium is regulated to as to supply the necessary and sufficient quantity for the cultivation of the microorganisms, with all CO₂ being consumed without loss into the atmosphere.

12. Method according to one of claims 1 to 11, characterised in that the only gas exchanges between the reactor and the external environment take place via the membrane contactor.

13. Method according to one of claims 1 to 12, characterised in that the microorganisms are bacteria, yeast, or protists, particularly microalgae.

14. Method according to one of claims 1 to 13, characterised in that the microorganisms are microalgae cultivated in autotrophic mode.

15. Use of a membrane contactor defined according to one of claims 1 to 13 to regulate the flows of CO₂ and O₂ in a method of cultivation of microorganisms, wherein the CO₂ is a source of carbon in the culture medium.

16. Use according to claim 15, characterised in that the method of cultivation comprises the use of a bioreactor comprising a culture medium.

17. Use according to claim 15, characterised in that the membrane contactor is placed in the bioreactor.

18. Microalgae fermentation device comprising a fermentation reactor, CO₂ supply means, and where applicable lighting means, characterised in that the CO₂ supply means comprise a membrane contactor for regulating the flows of CO₂ and O₂, wherein the flow of CO₂ is directed to the culture medium and the flow of O₂ is directed to the exterior of the culture medium.

19. Device according to claim 18, characterised in that the membrane contactor is defined according to one of claims 2 to 14.

20. Device according to one of claim 18 or 19, characterised in that the membrane contactor is placed in the fermentation reactor.
